(19) United States
(12) Patent Application Publication
(10) Pub. No.: US 2021/0303791 A1
(43) Pub. Date: Sep. 30, 2021

(54) FREE TEXT DE-IDENTIFICATION

(71) Applicant: KONINKLIJKE PHILIPS N.V., EINDHOVEN (NL)

(72) Inventors: DANIEL PLETEA, Eindhoven (NL); ROBERT PAUL KOSTER, Eindhoven (NL); PETER PETRUS VAN LIESDONK, EINDHOVEN (NL)

(21) Appl. No.: 17/260,265

(22) PCT Filed: Oct. 10, 2019
(86) PCT No.: PCT/EP2019/077500

§ 371 Date: Jan. 14, 2021

Related U.S. Application Data
(60) Provisional application No. 62/743,618, filed on Oct. 10, 2018.

(51) Int. Cl. G06F 40/289; G06K 9/00; G16H 10/60; G06F 40/30; G06F 40/166; G06F 40/268

(52) U.S. Cl. CPC G06F 40/289; G06K 9/00852; G16H 10/60; G06F 40/242; G06F 40/166; G06F 40/268; G06F 40/30

(57) ABSTRACT

A system or method generates de-identified output from a data set of patient data comprising unstructured text (100) in natural language phrases. A blacklist (105) has word items that are not allowed. The unstructured text is processed to determine a word count (110) comprising a list of low-rate word items that have a number of occurrences (k) in the unstructured text below a threshold (120). Subsequently, the low-rate word items and the blacklist word items are masked (130) in the unstructured text to generate the de-identified output (140).

### FIELD OF THE INVENTION

The present invention relates to the analysis of the handling of personally identifiable information (PII), such as patient data. More specifically, the present invention relates to the analysis and de-identification of patient data comprising free text, for example related to a disease or treatment. Such free text comprises natural language phrases and may include clinical notes, discharge summaries, handover notes, etc. and is called unstructured text in this document.

### BACKGROUND OF THE INVENTION

Recent regulations, e.g. GDPR “General Data Protection Regulation, Council of European Union, Regulation (eu) 2016/679 of the European parliament and of the council of 27 Apr. 2016 on the protection of natural persons with regard to the processing of personal data and on the free movement of such data, and repealing directive 95/46/ec, April 2016”, HIPAA “The health insurance portability and accountability act; U.S. Dept. of Labor, Employee Benefits Security Administration, 2004”, put strict requirements on the handling of personally identifiable information (PII), while also putting huge fines on noncompliance.

Text-based patient medical records are a vital resource in medical research and data analytics. In order to preserve patient privacy and confidentiality, regulation like the HIPAA and the GDPR require protected health information (PHI) to be removed from medical records before they can be used for secondary purposes. The de-identification of unstructured text documents is often realized manually and requires significant resources.

While there has been significant research done in the area of de-identification of structured clinical data (e.g. hospital databases, relational data warehouses), research on de-identifying data like free text clinical notes, discharge summaries and handover notes is less mature due to the unstructured nature of the data. Solutions for this problem use a multidisciplinary approach involving the domain knowledge on medical science, natural language processing (e.g. see “Hui Yang and Jonathan M. Garibaldi. Automatic detection of protected health information from clinic narratives. J. of Biomedical Informatics, 58(S):S30-S38, December 2015”), clinical text mining, machine learning (e.g. see “K. Rajput, G. Chetty, and R. Davey. Phis (protected health information) identification from free text clinical records based on machine learning; 2017 IEEE Symposium Series on Computational Intelligence (SSCI), pages 1-9, Nov 2017”) and recurrent neural networks (e.g. see “Franck Dernoncourt, Ji Young Lee, Ozlem Uzuner, and Peter Szolovits: De-identification of patient notes with recurrent neural networks; Journal of the American Medical Informatics Association, 24(3):596-606, 2017”).

However, blacklisting-based methods have a significant number of true-negatives due to the unstructured nature of the data. For example they cannot cover exceptions (e.g. “Summer” is both a name and a time indicator/season), misspellings (e.g. “Jonh” instead of “John”) or just the free nature of unstructured data (e.g. Christmas means actually December 25).

Additionally, de-identification of unstructured text is domain dependent and relies on domain specific dictionaries, which in most of the cases are not available. An example of such a domain specific dictionary is the MIMIC database (see “Ishna Neamatullah, Margaret M. Douglass, Li wei H. Lehman, Andrew T. Reisner, Mauricio Villarroel, William J. Long, Peter Szolovits, George B. Moody, Roger G. Mark, and Gari D. Clifford: Automated de-identification of free-text medical records; BMC Medical Informatics and Decision Making, 8:32-32, 2008”), while most of the other state-of-the-art de-identification methods rely on using blacklisting (e.g. see “Stéphane M. Meystre, F. Jeffrey Friedlin, Brett R. South, Shuying Shen, and Matthew H. Samore: Automatic de-identification of textual documents in the electronic health record: a review of recent research; BMC medical research methodology, 2010”).

Machine learning techniques need training data, which in addition needs to be annotated. Such requirements may be hard to satisfy at least in a short time manner and would need to be repeated for different domains. Furthermore the amount of data that is needed for training is a lot bigger than for example just a simple one time de-identification task.

However, current free-text de-identification methods do not mask identifiers that are not covered by blacklists, and also have the following problems:
Domain language. De-identification of unstructured text may require knowledge of the domain (e.g. MIMIC database, domain-specific words) and in many cases domain-based white-lists are not available due to not being yet built. De-identification experts may also be slowed down by the specificity of the domain.
True-negatives. Misspellings are part of the PHI that should be masked in the de-identified output, but they are slipping the usual methods of de-identification.
Inefficiency. Current methods need building domain knowledge and white-listing based on manual review. The de-identification of unstructured text documents is often realized manually, and requires significant resources.

### SUMMARY OF THE INVENTION

It is an object of the invention to provide a method and system for free text de-identification that takes into account at least one of the preceding issues.

For this purpose, devices and methods for generating de-identified output from a data set of patient data are provided as defined in the appended claims. According to an aspect of the invention a method for generating de-identified output from a data set of patient data of multiple patients is provided as defined in claim 1. A system is provided as defined in claim 13. According to a further aspect of the invention there is provided a computer program product downloadable from a network and/or stored on a computer-readable medium and/or microprocessor-executable medium, the product comprising program code instructions for implementing the above method when executed on a computer.

To overcome these disadvantages, the de-identification method for unstructured text masks or removes (blacks out) word items which do not occur often in the text and blacklisted word items. Thereto the unstructured text is de-identified by performing a word count and allowing in the de-identified output only words occurring in the text more than a minimum number of occurrences. The method further suppresses or replaces words that are blacklisted (e.g. the 18 HIPAA Identifiers). The word count provides a list of low-rate word items that have a number of occurrences (k) in the unstructured text below a threshold. Then, the low-rate word items and the blacklist word items are removed from, or masked, in the unstructured text to generate the de-identified output. Word items may include, next to words as-is, word sequences, word stems, and word patterns.

Advantageously, the method and system do not require initial domain knowledge input and are able to lower the amount of true-negatives in comparison with state of the art solutions.

In an embodiment of the invention the word items in the word-count and/or blacklist entries are associated to the syntactic category (verb, noun, etc.) that the word has in the text, as determined by natural language processing (NLP). This increases the quality of the blacklist by discovering words that are potential identifiers, but not covered by the blacklist due to known limitations of static blacklists.

In another embodiment of the invention a domain-specific white-list word list is created from the words that passed the word count. These words can be later allowed in the de-identified output even if in some cases their occurrence is not high.

The methods according to the invention may be implemented on a computer as a computer implemented method, or in dedicated hardware, or in a combination of both. Executable code for a method according to the invention may be stored on a computer program product. Examples of computer program products include memory devices such as a memory stick, optical storage devices such as an optical disc, integrated circuits, servers, online software, etc.

The computer program product in a non-transient form may comprise non-transitory program code means stored on a computer readable medium for performing a method according to the invention when said program product is executed on a computer. In an embodiment, the computer program comprises computer program code means adapted to perform all the steps or stages of a method according to the invention when the computer program is run on a computer. Preferably, the computer program is embodied on a computer readable medium. There is also provided a computer program product in a transient form downloadable from a network and/or stored in a volatile computer-readable memory and/or microprocessor-executable medium, the product comprising program code instructions for implementing a method as described above when executed on a computer.

Another aspect of the invention provides a method of making the computer program in a transient form available for downloading. This aspect is used when the computer program is uploaded into, e.g., Apple's App Store, Google's Play Store, or Microsoft's Windows Store, and when the computer program is available for downloading from such a store.

Further preferred embodiments of the devices and methods according to the invention are given in the appended claims, disclosure of which is incorporated herein by reference.

### BRIEF DESCRIPTION OF THE DRAWINGS

These and other aspects of the invention will be apparent from and elucidated further with reference to the embodiments described by way of example in the following description and with reference to the accompanying drawings, in which

FIG. 1 shows a schematic flow chart illustrating an embodiment of the method for de-identification of a set of patient data containing unstructured text,

FIG. 2 shows a schematic flow chart illustrating an embodiment of the method for de-identification of a set of patient data containing natural language processing of the unstructured text,

FIG. 3 shows a schematic flow chart illustrating an embodiment of the method for de-identification of unstructured text using a whitelist,

FIG. 4 shows a schematic flow chart illustrating an embodiment of the method for de-identification of unstructured text using a confidence list,

FIG. 5 shows an embodiment of the method for de-identification of a set of patient data containing unstructured text,

FIG. 6a shows a computer readable medium, and

FIG. 6b shows in a schematic representation of a processor system.

The figures are purely diagrammatic and not drawn to scale. In the Figures, elements which correspond to elements already described may have the same reference numerals.

### DETAILED DESCRIPTION OF EMBODIMENTS

The present invention will be described with respect to particular embodiments and with reference to the figures, but the invention is not limited thereto, but only to the claims.

The term “individual” refers to a human subject. Said human subject may or may not be affected by or suffering from a disease to be studied. Hence, the terms “individual”, “person” and “patient” are synonymously used in the instant disclosure.

The expression “providing patient data” is understood that the patient data of at least one individual need to be obtained. However, the patient data of the at least one individual do not have to be obtained in direct association with the method or for performing the method. Typically the patient data of the at least one individual are obtained at a previous point or period of time, and are stored electronically in a suitable electronic storage device and/or database. For performing the method, the patient data can be retrieved from the storage device or database and utilized.

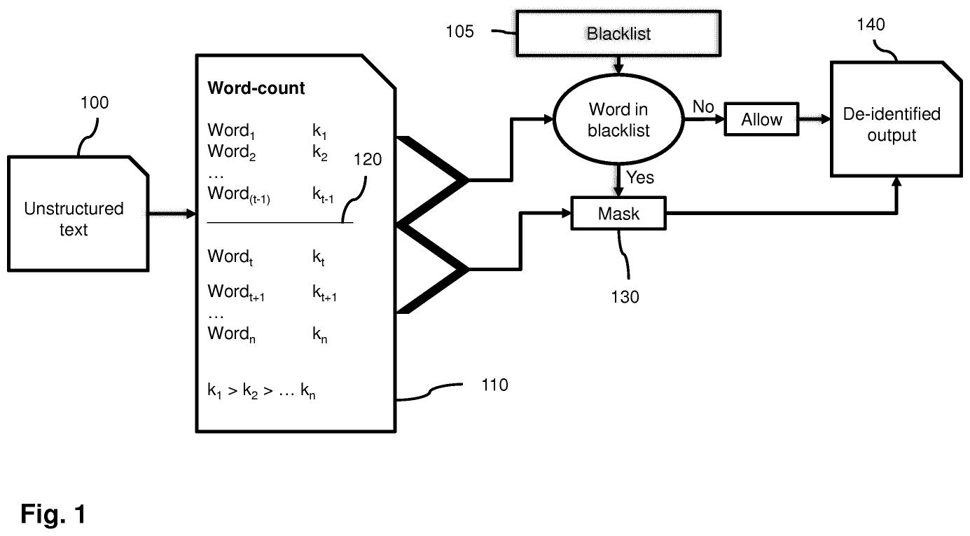

FIG. 1 shows a schematic flow chart illustrating an embodiment of the method for de-identification of a set of patient data containing unstructured text. This method will typically be implemented as a software framework that makes the whole process practical to use. The Figure depicts a computer-implemented method for generating de-identified output from a data set of patient data of multiple patients. The patient data includes unstructured text 100. The unstructured text consists of word items, such as words, numbers and symbols, arranged in natural language phrases. A blacklist 105 has blacklist word items that are not allowed in the de-identified output.

In a first phase, the method processes the unstructured text to determine a word count 110. The word count has a list of low-rate word items that have a number of occurrences (k) in the unstructured text below a threshold 120, schematically indicated by a line separating the low-rate word items (kₜ to kₙ) form the word items that occur more often than the threshold. In a second phase the method removes or masks 130 the low-rate word items in the unstructured text to generate the de-identified output 140. Also, the word items are masked (when they are in the blacklist) or allowed (when they are not in the blacklist).

The blacklist may be designed to find the HIPAA 18 Identifiers. Thereto the blacklist may be compound and may include dictionaries (e.g. names) and regular expressions for zip codes, dates, emails, URLs, IP addresses and the rest of unique identifying numbers (e.g. driver license). Even with such an extensive list of regular expression, blacklists have their limitations. For example they cannot cover exceptions (e.g. “Smart” can be both a name and an adjective), misspellings (e.g. “Jonh” instead of “John”) or just the free nature of unstructured data (e.g. Christmas means actually December 25). Such examples would have a small number of occurrences in the full text, below the threshold and therefore be masked in the de-identified output.

The threshold may be set statically by setting it on a number T considered safe by a de-identification expert. Also, the threshold may be set dynamically, by going through the words in the word-count list until at least a desired percentage P % of the text is allowed in the de-identified output. This should happen without passing the minimum static threshold described above. So, the processing may include setting the threshold above a minimum threshold in dependence of a desired percentage of the unstructured text that is allowed in the de-identified output.

The “Word-count” list may be the result of a simple operation of counting only word items, such as words as-is.

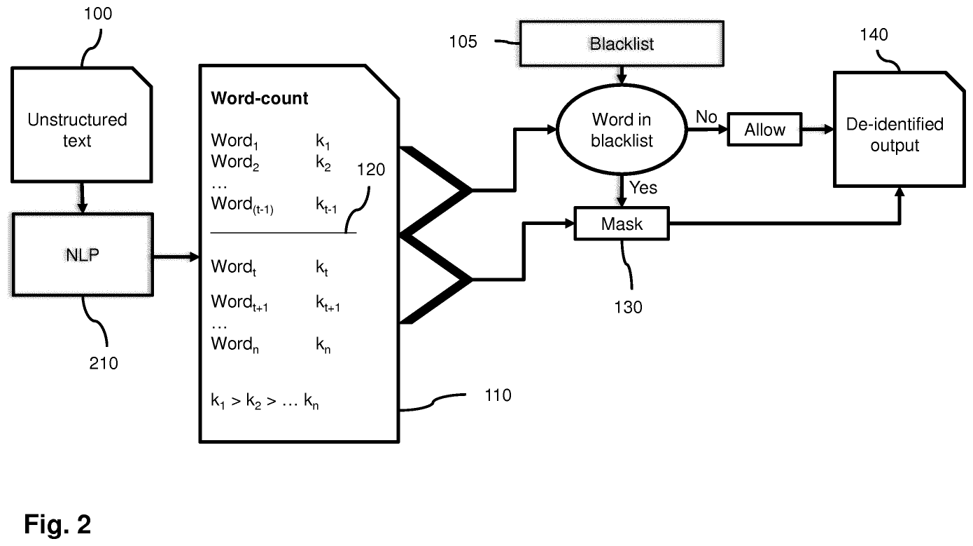

FIG. 2 shows a schematic flow chart illustrating an embodiment of the method for de-identification of a set of patient data containing natural language processing of the unstructured text. The word count may include a preprocessing 210 of the unstructured text, generally called natural language processing, e.g. see “Steven Bird, Ewan Klein, and Edward Loper: Natural Language Processing with Python; O'Reilly Media, Inc., 1st edition, 2009”. Various embodiments of such processing, which may be combined, are described now.

Optionally, the method may include determining, as word items, separate word items for a same word having different syntactic positions in the phrases. The natural language processing 210 may be arranged for factoring in the syntactic position of the words as depicted in FIG. 2. The word syntactic position may, for example, be noun phrase; verb phrase; prepositional phrase; determiner; noun; verb; preposition. For example the word “smart” is used in the sentence “Smart went to the store” as a noun, while in the sentence “Smart kids went to the store” it is used as an adjective. “smart” used as an adjective will have many more occurrences than “smart” used as a noun and therefore the noun entry will be masked. Furthermore, the word count or syntactic position may not consider if the words start with capital or not. However, when words are used as a noun, a capital may be indicative of a name, and such word items may be separately counted.

Optionally, the method may include determining, as word items, word patterns, a word pattern comprising in a phrase at least one word in combination with an adjacent pattern of numbers or symbols. The Natural Language Processing 210 may be arranged for determining patterns, such as: “[0-9]+word” or “[0-9]+word” where the decision of allowing or masking is based on the word within the word item. This way “Monday 11:00” will be allowed, while “January 23” will be masked because “Monday” is not blacklisted, while “January” is blacklisted based on the HIPAA 18 identifiers being a date.

Optionally, the method may include determining, as word items, word strings, a word string comprising a specific sequence of words. The Natural Language Processing 210 may be arranged for determining word combinations, short sequences or small sentences. Such strings may be determined automatically as longest repeating strings, where the number k of occurrences is higher than the threshold.

The above options may be combined. So the processing may include determining the blacklist using the word items as defined above.

Optionally, the method may include determining, as word items, word stems, a word stem being a set of different words having a similar semantic function in different phrases. The Natural Language Processing 210 may be arranged for detecting and combining such different words to be counted together. For example, word stems of a verb, e.g. “was”, “is”, “were” are all part of the “to be” class, may cover more words that should be allowed in the de-identified output.

The above options may be combined. So the processing may include determining the word count using the word items as defined above.

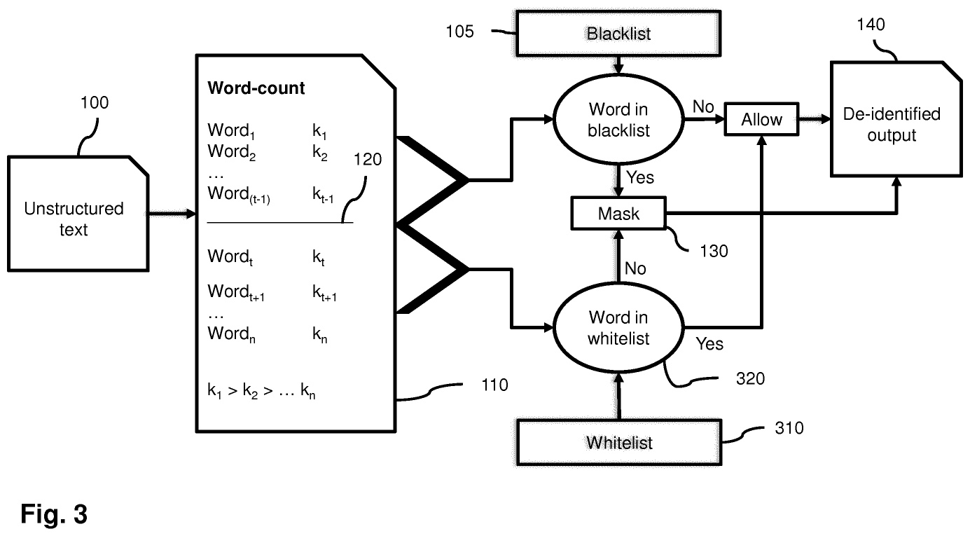

FIG. 3 shows a schematic flow chart illustrating an embodiment of the method for de-identification of unstructured text using a whitelist. The word items from the whitelist can be later allowed in the de-identified output even if their occurrence does not exceed the threshold. The whitelist may be general, or built for a respective domain, or may be generated in time based on past de-identification events.

In the Figure, the processing depicts determining a whitelist 310 comprising word items that are allowed in the de-identified output. Also by whitelist test 320, said removing or masking the low-rate word items is prevented by allowing in the de-identified output low-rate word items that are in the whitelist. So, a domain-specific white-list may be created of word items are allowed even if they do no pass the word count criterion, i.e. low-rate word items. FIG. 3 shows a first way of using the whitelist 310. Any low-rate words that are detected in the word-count are tested for being in the whitelist. If so, the word is allowed in the de-identified output 140, and if not, the word is masked 130.

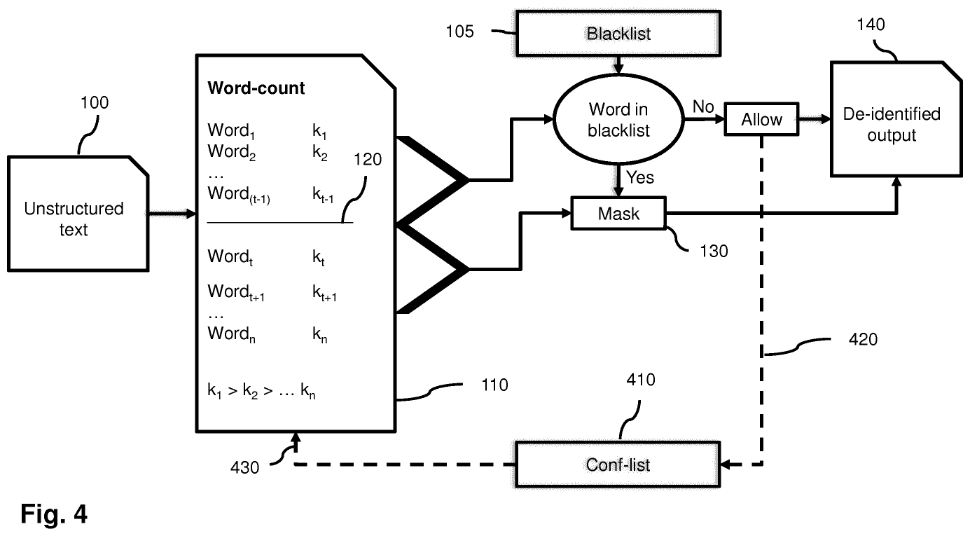

FIG. 4 shows a schematic flow chart illustrating an embodiment of the method for de-identification of unstructured text using a confidence list. The confidence list in the embodiment may be a domain-specific list built in time. In the Figure, the processing comprises determining a confidence list 410 comprising a confidence score for confidence word items based on word count results in previous de-identification events, as indicated by a dashed arrow 420. Also, the Figure shows a dashed arrow 430 indicating adapting the word count. The word count is adapted for the confidence word items by adjusting, in dependence of the confidence score, the number of occurrences (k) or the threshold.

The words in the confidence list 410 (which may be general or determined for a respective domain) have a confidence score ConfScore. Optionally, the confidence score represents a percentage how many times the confidence word item was above the threshold in the word count in the previous de-identification events. Adapting the word-count may involve adapting k in the “Word-count” list using the ConfScore, which for example becomes k=k*ConfScore. The initial value of the ConfScore for a word not yet existing in the Whitelist (domain) would be 1, and would be higher than 1 depending on the number of occurrences if the word was allowed in earlier de-identification events. Alternatively the threshold could be lowered based on the ConfScore, and/or the ConfScore could be normalized.

In the various embodiments blacklisting is combined with masking low-rate word-items based on the word-count. So, in addition to the removing the blacklisted words items such as HIPAA 18 identifiers, the proposed method is removing outliers which occur less times than the threshold. For example the “Lamborghini” word would have a small occurrence in the full text the text “I picked the kids with the Lamborghini in my way to the hospital”. The proposed method would mask the “Lamborghini” word by suppressing or replacing it.

The method as presented in the Figures has been tested on a dataset comprising of 6670 different words (239218 in total). The threshold T was set to a value of 10 minimum occurrences. The results are the following:
Allowed text: 95%;
7 allowed domain words from medical domain;
3 masked names (1 occurrence each);
Masked months examples: June (3 occurrences), September (1 occurrences).

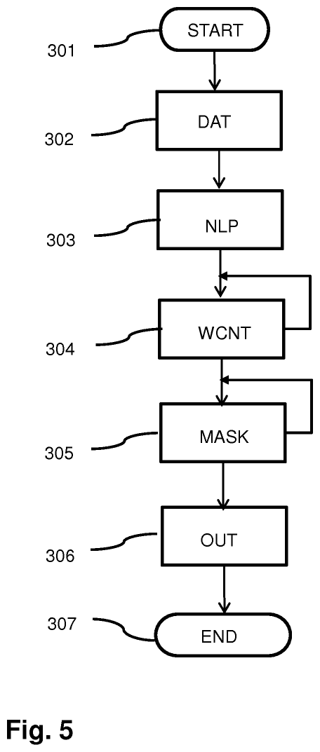

FIG. 5 shows an embodiment of the method for de-identification of a set of patient data containing unstructured text. The Figure depicts a computer-implemented method for generating de-identified output from a data set of patient data of multiple patients.

The method starts at node START 301, and step DAT 302 represents obtaining, e.g. collecting and storing, a set of patient data of multiple individuals. The patient data includes unstructured text. The unstructured text consists of word items, such as words, numbers and symbols, arranged in natural language phrases. Also, a blacklist is obtained that has blacklist word items that are not allowed in the de-identified output.

Optionally, in a preprocessing step NLP 303 natural language processing is performed on the unstructured text to identify word items, like syntactic word positions, word strings, word patterns and word stems as discussed above.

In a first process word-count WCNT 304, the method processes the unstructured text to determine a word count. The word count has a list of low-rate word items that have a number of occurrences in the unstructured text below a threshold.

In a second process MASK 305 the method processes the unstructured text to remove or mask the low-rate word items in the unstructured text. Also, the blacklist is applied: word items are masked (when they are in the blacklist) or allowed (when they are not in the blacklist). Finally in step OUT 306, the method generates the de-identified output. Then the method terminates in step END 307.

The above methods may be applied to de-identify any unstructured text data independent of the domain of the data. They may be used for making available de-identified medical data for secondary purposes as research and data analytics, for example, in heath data analysis platform or similar platforms. They may also be used as a client application that interacts with a data-lake for making available data to its clients. Furthermore, the methods may be applied on any form of privacy preserving computation that results in a dataset that still contains personal information and any data export, e.g. for research. In an embodiment, the method may be used in diagnostics, wherein the genetic information of an individual is analyzed for the genetic disposition and/or occurrence of a specific disease or disorder of said individual.

The methods may be applied to any disease, disorder or medical condition. A disease to be studied may be a specific disease that is chosen on purpose. In an embodiment, the disease to be studied is known to be a disease that is associated with a particular genotype. Examples of such diseases are cancers, immune system diseases, nervous system diseases, cardiovascular diseases, respiratory diseases, endocrine and metabolic diseases, digestive diseases, urinary system diseases, reproductive system diseases, musculoskeletal diseases, skin diseases, congenital disorders of metabolism, and other congenital disorders such as prostate cancer, diabetes, metabolic disorders, or psychiatric disorders.

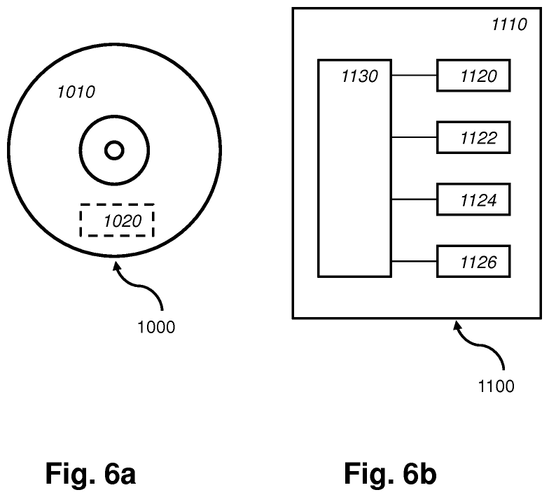

In an embodiment, the methods as described with FIGS. 1 to 4 may be implemented in a system 1100 as depicted in FIG. 6b, discussed later, e.g. on a computer as a computer implemented method, as dedicated hardware, or as a combination of both. As also illustrated in FIG. 6a, instructions for the computer, e.g., executable code 1020, may be stored on a computer readable medium 1000, e.g., in the form of a series of machine readable physical marks and/or as a series of elements having different electrical, e.g., magnetic, or optical properties or values. The executable code may be stored in a transitory or non-transitory manner. Examples of computer readable mediums include memory devices, optical storage devices, integrated circuits, servers, online software, etc. The Figure shows an optical disc 1010.

It will be appreciated that the invention applies to computer programs, particularly computer programs on or in a carrier, adapted to put the invention into practice. The program may be in the form of a source code, an object code, a code intermediate source and an object code such as in a partially compiled form, or in any other form suitable for use in the implementation of the method according to the invention. It will also be appreciated that such a program may have many different architectural designs. For example, a program code implementing the functionality of the method or system according to the invention may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g. Java interpreter instructions). Alternatively, one or more or all of the sub-routines may be stored in at least one external library file and linked with a main program either statically or dynamically, e.g. at run-time. The main program contains at least one call to at least one of the sub-routines. The sub-routines may also comprise function calls to each other. An embodiment relating to a computer program product comprises computer-executable instructions corresponding to each processing stage of at least one of the methods set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

FIG. 6a shows a computer readable medium 1000 having a writable part 1010 comprising a computer program 1020, the computer program 1020 comprising instructions for causing a processor system to perform one or more of the above methods and processes in the system as described with reference to FIGS. 1-4. The computer program 1020 may be embodied on the computer readable medium 1000 as physical marks or by means of magnetization of the computer readable medium 1000. However, any other suitable embodiment is conceivable as well. Furthermore, it will be appreciated that, although the computer readable medium 1000 is shown here as an optical disc, the computer readable medium 1000 may be any suitable computer readable medium, such as a hard disk, solid state memory, flash memory, etc., and may be non-recordable or recordable. The computer program 1020 comprises instructions for causing a processor system to perform said methods.

FIG. 6b shows in a schematic representation of a processor system 1100 according to an embodiment of the devices or methods as described with reference to FIGS. 1-5. The processor system may comprise a circuit 1110, for example one or more integrated circuits. The architecture of the circuit 1110 is schematically shown in the Figure. Circuit 1110 comprises a processing unit 1120, e.g., a CPU, for running computer program components to execute a method according to an embodiment and/or implement its modules or units. Circuit 1110 comprises a memory 1122 for storing programming code, data, etc. Part of memory 1122 may be read-only. Circuit 1110 may comprise a data interface 1126, comprising, e.g., an antenna, a transceiver for internet, connectors or both, and the like. Circuit 1110 may comprise a dedicated integrated circuit 1124 for performing part or all of the processing defined in the method. Processor 1120, memory 1122, dedicated IC 1124 and communication element 1126 may be connected to each other via an interconnect 1130, say a bus. The processor system 1110 may be arranged for wired and/or wireless communication, using connectors and/or antennas, respectively.

The system 1100 is configured to anonymizing patient data as described with the above methods, e.g. elucidated with reference to FIG. 3. The system comprises a data interface 1126 configured to access patient data of multiple individuals. The data interface may be in communicative with database on a local storage unit or on a server. The data interface may be connected to an external repository, such as a suitable electronic storage device and/or database, which comprises the patient data. Alternatively, the patient data or a database may be accessed from an internal data storage of the system 1122. In general, the data interface may take various forms, such as a network interface to a local or wide area network, e.g., the Internet, a storage interface to an internal or external data storage, etc.

Furthermore, the system 1100 may have a user input interface configured to receive user input commands from a user input device to enable the user to provide user input, such as choose or define a particular disease, disorder or medical condition for subsequently determining a subset of patient data being related to said disease, disorder or medical condition. The user input device may take various forms, including but not limited to a computer mouse, touch screen, keyboard, etc.

It will be appreciated that, for clarity, the above description describes embodiments of the invention with reference to different functional units and processors. However, it will be apparent that any suitable distribution of functionality between different functional units or processors may be used without deviating from the invention. For example, functionality illustrated to be performed by separate units, processors or controllers may be performed by the same processor or controllers. Hence, references to specific functional units are only to be seen as references to suitable means for providing the described functionality rather than indicative of a strict logical or physical structure or organization. The invention can be implemented in any suitable form including hardware, software, firmware or any combination of these.

According to a further aspect, the invention concerns the use of the method and/or the computer program product in research and/or in diagnosis. In an embodiment, the method and/or computer program product is used in bioinformatics research. The use of the method, system and/or computer program product in bioinformatics research comprises acquisition the patient data of a plurality of individuals. Examples of research fields are genomics, genetics, transcriptomics, proteomics and systems biology.

In an alternative embodiment, the method, system and/or computer program product may be used in diagnosis, wherein the patient data of an individual are utilized to analyze whether the individual is affected by a specific disease or at risk of getting said disease or being affected by said disease. The individuals are sure that their patient data are properly anonymized.

Where an indefinite or definite article is used when referring to a singular noun, e.g. “a”, “an”, “the”, this includes a plural of that noun unless something else is specifically stated. Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Moreover, the terms top, bottom, over, under, beyond and the like in the description and in the claims are used for descriptive purposes and not necessarily for describing relative positions. It is to be understood that the terms so used are interchangeable under appropriate circumstances and that the embodiments of the invention described herein are capable of operation in other orientations than described or illustrated herein. It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A computer-implemented method for generating de-identified output from a data set of patient data of multiple patients,
the patient data comprising unstructured text, the unstructured text comprising word items of words, numbers and symbols arranged in natural language phrases, and
a blacklist comprising blacklist word items that are not allowed in the de-identified output,
the method comprising the steps of:
processing the unstructured text to determine a word count comprising a list of low-rate word items that have a number of occurrences (k) in the unstructured text below a threshold, and
removing or masking the low-rate word items and the blacklist word items in the unstructured text to generate the de-identified output.

2. The method according to claim 1, wherein the processing comprises setting the threshold above a minimum threshold in dependence of a desired percentage of the unstructured text that is allowed in the de-identified output.

3. The method according to claim 1, wherein the method comprises determining, as word items, separate word items for a same word having different syntactic positions in the phrases.

4. The method according to claim 1, wherein the method comprises determining, as word items, word patterns, a word pattern comprising in a phrase at least one word in combination with an adjacent pattern of numbers or symbols.

5. The method according to claim 1, wherein the method comprises determining, as word items, word strings, a word string comprising a specific sequence of words.

6. The method according to claim 1, wherein the method comprises determining, as word items, word stems, a word stem comprising a set of different words having a similar semantic function in different phrases.

7. The method according to claim 1, wherein the processing comprises determining the blacklist using the word items as claimed in claim 1.

8. The method according to claim 1, wherein the processing comprises determining the word count using the word items as claimed in claim 1.

9. The method according to claim 1, wherein the processing comprises
determining a whitelist comprising word items that are allowed in the de-identified output, and
preventing said removing or masking the low-rate word items by allowing in the de-identified output low-rate word items that are in the whitelist.

10. The method according to claim 1, wherein the processing comprises
determining a confidence list comprising a confidence score for confidence word items based on word count results in previous de-identification events, and
adapting the word count for the confidence word items by adjusting, in dependence of the confidence score, the number of occurrences (k) or the threshold.

11. The method according to claim 10, wherein the confidence score represents in a percentage how many times the confidence word item was above the threshold in the word count in the previous de-identification events.

12. A computer program product for generating de-identified output from a data set of patient data of multiple patients, the computer program product comprising instructions which when carried out on a computer cause the computer to perform a method as claimed in claim 1.

13. A system for generating de-identified output from a data set of patient data of multiple patients, the system comprising:
a data interface configured to receive patient data of multiple patients, the patient data comprising unstructured text, the unstructured text comprising word items of words, numbers and symbols arranged in natural language phrases, and
a blacklist comprising blacklist word items that are not allowed in the de-identified output; and
a processor arranged to:
process the unstructured text to determine a word count comprising a list of low-rate word items that have a number of occurrences (k) in the unstructured text below a threshold, and
remove or mask the low-rate word items and the blacklist word items in the unstructured text to generate the de-identified output.

14. Use of the method according to claim 1, the computer program product and/or the system in one selected from the group consisting of genomics, genetics, bioinformatics research, transcriptomics, proteomics and systems biology or diagnosis.
